(19) United States
(12) Patent Application Publication
(10) Pub. No.: US 2010/0260830 A1
(43) Pub. Date: Oct. 14, 2010

(54) Liposomal Formulations of Tocopheryl Amides

(76) Inventors: Brian A Salvatore, Shreveport, LA (US); Jaroslav Turanek, Brno (CZ); Jiri Neuzil, Runaway Bay (AU)

Correspondence Address: K. Karel Lambert, P.O. Box 4063, Renton, WA, 98057, US

(21) Appl. No.: 12/755,649

(22) Filed: Apr. 7, 2010

(30) Foreign Application Priority Data
Apr. 8, 2009 (AU) AU 2009/901523

(51) Int. Cl. A61K 9/127; A61K 31/355; A61P 35/00

(52) U.S. Cl. 424/450; 514/458; 977/907

(57) ABSTRACT

Formulations of N-chroman dicarboxylic acid derivatives and their bioisosteres in liposomal systems. Lyophilized liposomal dosage forms of N-chromans, are found to be stable, to achieve therapeutically meaningful plasma levels on administration to a mammalian host, and to demonstrate selective pro-apoptotic oncolytic properties in vivo. Advantageously, these formulations overcome the systemic toxicity that characterized their administration by other dosage forms.

### CROSS-REFERENCE TO RELATED APPLICATIONS

This application claims priority to Australian Provisional Patent Application AU 2009/901523, filed Apr. 8, 2009 and titled “Liposomal Formulations of Tocopheryl Amides”, from which is priority claimed, and which is herein incorporated in full by reference.

### FIELD OF THE INVENTION

This invention is related to liposomal formulations of N-chromanyl amino hemi-dicarboxylic acid amides, their bioisosteres, and chromanols more generally. Such compounds have demonstrated oncolytic properties in vitro and in vivo, and the formulations are found to be useful for administration to a mammalian host in need thereof.

### BACKGROUND

Vitamin E analogues have received increasing interest as oncolytics. The recently discovered in vitro potency of N-chromanyl amino hemi-dicarboxylic acid amides has prompted interest their formulation for clinical use. These compounds are characterized by a carboxylic acid moiety attached to an amine substituent on the polar head of the chroman nucleus and a tocopherol-like hydrophobic tail, and include bioisosteres thereof. However, the compounds have been found to be systemically toxic in vivo when delivered in conventional systems. Challenges in clinical use of these new oncolytic agents include their very low solubility in aqueous systems and the instability of biphasic systems as might be useful in their formulation. Thus far, no formulations of pro-apoptotic N-chromans suitable for parenteral injection or infusion have been reported.

Delivery systems for tocopheryl-succinate esters provide some experience. Tocopheryl succinate has been solubilized in organic solvents such as ethanol and dimethylsulphoxide (DMSO) or in oil emulsions, spontaneously vesiculated, conjugated with polyethylene glycol, and formulated as pH sensitive liposomes (Jizomoto et al, 1994, pH-Sensitive liposomes composed of tocopherol hemisuccinate and of phosphatidylethanolamine including tocopherol hemisuccinate, Biochim Biophys Acta 1213:343-48). However, these liposomal systems resulted in high uptake by the reticuloendothelial system (approaching 70% in liver and spleen), and were relatively unstable. The use of phosphatidylethanolamine resulted in pH-gated release of the drug anion from the vesicles.

Tocopheryl succinate (αTOS), when formulated in unilamellar liposomes prepared with phosphatidylcholine and bile salts by the detergent method, was found to be effective against a cell line in vitro (Gu et al, 2006. Cytotoxicity of liposomal α-tocopheryl succinate toward hamster cheek pouch carcinoma (HCPC-1) cells in culture, Cancer Lett 239:281-91). However, use of bile acids in clinical formulations is likely associated with hemolysis and toxicity on intravenous injection and is preferably avoided.

Chan, in US Pat. Appl. No. 2004/0175415, proposed administration of tocopheryl succinate to humans as a transdermal cream prepared with almond oil and dimethylsulfoxide, but these formulations were abandoned, and are associated with spontaneous and erratic formation of mixed micelles or emulsions when contacted with bodily fluids and are unsuitable for injectables.

Ralph, in WO 2008/031171 also proposed formulations of tocopheryl succinate. Claims to transdermal formulations of pro-apoptotic formulations are presented, and the reported vehicle is a transdermal gel for treating mesothelioma, where the gel is a commercially available product termed “Lipoderm”, which consists of a PLO form of Poloxamer (Pluronic F127) surfactant as gel-forming hydroalcohol, isopropyl palmitate as solvent and penetration enhancer, and lecithin as a co-surfactant, which the authors incorrectly characterize as a liposome, or a PCCA form discussed below. Literature reports indicate that lecithin and isopropyl palmitate in a ratio of 1:1 must comprise 24% of the system in order for micelles containing the drug to form properly (Boothe, D M, “The revolving concerns of dispensing and compounding”, accessed on-line 2010 Apr. 4). The formulation is thus more properly considered an emulsion or microemulsion precursor. The gels are not liquid at temperatures below 40° C. and because of the high surfactant concentrations, are not suitable for intravenous use. Interestingly, the gels again become liquid under refrigeration, a property recognized by those familiar with solutions of Pluronic F127, and may not be freeze dried. Furthermore, a review of the scientific literature reveals limited scientific support of the use of the PLO gel system in delivering drugs, although a variety of weight loss compounds are marketed and there is anecdotal evidence for their use. Allergies to plant or egg lecithins may develop with continued topical use. Lipoderm® in PCCA form is the subject of US Pat. Appl. 2009/0285869 to Trimble but is again a transdermal cream containing lecithin, isopropyl palmitate and non-Pluronic emulsifiers, and is not suited for intravenous use for a variety of reasons: 1) Biphasic formulations in general are plagued by chemical instability of the active in the aqueous compartment 2) and by physical instability. Generally, excess surfactant is used to physically stabilize the product, 3) increasing the toxicity of the vehicle. Emulsions that are not terminally sterilizable 4) must be processed under sterile conditions, complicating their utility for injectables. These formulations are readily differentiated from liposomes, as is well known in the art. Trimble characterizes the gels as emulsions, stating: “Lecithin organogel (LO) is composed of emulsifiers, hydrocolloids, and lecithin. It is a second generation pluronic lecithin organogel (“PLO”). The pluronic component has been removed. The advantages of the LO compared to the original PLO are that it is non-greasy, non-tacky, has improved stability to temperature, and has improved stability to salts. The emulsion phase may be prepared by adding oil-in-water (O/W) and water-in-oil (W/O) emulsifiers to oil and agitating periodically to ensure complete dissolution.” (para 0037-38).

Using a combination of vesiculated αTOS and dendritic cell-based immunotherapy, successful experimental inhibition of tumor growth and metastasis of an aggressive murine mammary cancer cell line 4T1 was demonstrated (Ramanathapuram et al, 2005, Chemo-immunotherapy of breast cancer using vesiculated α-tocopheryl succinate in combination with dendritic cell vaccination, Nutr Cancer 53:177-93). Vesicles were prepared by sonicating rehydrated αTOS with NaOH in phosphate-buffered saline. Kogure (Kogure K et al, 2005, Cytotoxicity of α-tocopherol succinate, malonate and oxalate in normal and cancer cells in vitro and their anti-cancer effects on mouse melanoma in vivo, J Nutr Sci Vitaminol 51:392-97; Kogure K et al, Potentiation of anti-cancer effect by intravenous administration of vesiculated α-tocopherol hemisuccinate, Cancer Lett 192:19-24) also described self-vesiculated preparations of tocopheryl succinate and related chromanols prepared by sonication with NaOH. However, this general approach, when applied in our hands to the more potent N-chromans, was found to result in vascular and systemic toxicity and was unworkable for preclinical studies, impeding further research.

Thus there remains a need for formulations useful in advancing pro-apoptotic N-chroman compounds to the clinic. The liposomal formulations of the present invention, surprisingly, relieve the non-specific toxicity encountered when these compounds were tested initially in vivo. The liposomal formulations are unique in that the vesicle bilayer is formed of a mixture of a carrier phospholipid or polar liposome-forming lipid and a therapeutic, pro-apoptotic vitamin E analogue and are processed to minimize the aqueous compartment and preserve the oligolamellar character of the hydrated mixture. These formulations and methods have applicability to N-chromans, and to chromans in general, relieving the non-specific toxicity of certain of the compounds, and also address the related problems of stability and manufacturability necessary for therapeutic use of these compounds.

### SUMMARY

The invention relates to formulations needed to advance highly pro-apoptotic, oncolytic N-chromans to clinical trials. The mixed liposomal formulations of the present invention, surprisingly, address the non-specific toxicity encountered when these compounds were first tested in vivo. In earlier in vitro studies, the toxicity of the compounds was generally specific for cancer cells and not fibroblasts or other normal cell types, but paradoxically, solutions of the N-chroman compounds tested in ethanol or DMSO were generally toxic or fatal when injected in efficacious doses in animals. Similarly toxic were vesicular preparations of N-chromans prepared by self-vesiculation. Those vesicles were associated with severe toxicity—both localized vascular toxicity and generalized systemic toxicity—upon injection, impeding further research. Surprisingly however, by formulating the compounds as a mixed liposomal formulation with a polar lipid such as a lecithin, efficacious doses by injection were readily achieved. The role of the carrier lipid in solving the problem of toxicity on injection is not fully understood.

Paradoxically and unexpectedly, we have found that the liposomal formulations of the invention overcome the problematic in vivo toxicity of the compounds—while enhancing the tumor-specific cytocidal pro-apoptotic action of the compounds.

The liposomes of the invention are unique in that the vesicle bilayer is formed of a mixture of a phospholipid, or other polar liposome-forming lipid, and a pro-apoptotic N-chroman or chromanol. The liposomal structures of the present invention may be characterized by mixed polar lipid contents, where an N-chroman or chromanol is dispersed in a bilayer of a carrier lipid such as a lecithin. These formulations and methods have applicability to N-chromans, and to chromans in general.

Thus the liposomes of the present invention are unusual in that the efficacious compounds are not strictly “encapsulated” for delivery, but when co-processed with a phospholipid, are co-mingled in the lipid bilayers of multilamellar vesicles and are thought to be available for sustained release. Surprisingly, the presence of the phospholipid in these formulations has a dramatic moderating effect on non-specific toxicity while not sacrificing the pro-apoptotic oncolytic properties of these compounds. In a preferred embodiment, the formulation methods are optionally modified by omission of the freeze-thaw intermediate process step prior to lyophilization so as to minimize the aqueous compartment and preserve the oligolamellar character of the mixed liposomal product, improving both stability and sustained release of the actives.

Advantageously, when formulated with a lyoprotectant excipient such as sucrose or trehalose, the liposomal formulations of the invention may be dehydrated for storage in dry form, and upon reconstitution are shown to be physically and chemically stable, with full biological activity. Thus in a first embodiment the invention is a lyophilized multilamellar liposomal formulation of a pro-apoptotic chroman, comprising an N-chromanyl amino hemi-dicarboxylic acid amide (or a bioisostere thereof) in a molar ratio of up to 30:70 with a phospholipid, preferably a lecithin, and a lyoprotectant excipient, where the formulation, when lyophilized and reconstituted with a physiologically compatible diluent, forms a monodisperse liposomal suspension suitable for intravenous infusion, for intratracheal infusion, or for injection, while not limited thereto.

Chroman-containing liposomes of the invention are prepared for example by a proliposome-liposome method, by hydration of a lipid film followed by extrusion through polycarbonate filters, or by other established industrial apparatus for extrusion, homogenization, or microfluidization. Larger volumes are processed by extrusion using a high pressure cell as described by Turanek (1994. Fast-protein liquid chromatography system as a tool for liposome preparation by the extrusion procedure, Anal Biochem 218:352-357), for example. Polydispersity indices are generally less than 0.15 with increased dose loading; zeta potentials are more negative than −10 mV at neutral pH. Mean size distributions of less than 180 nm are readily achieved, indicating that the formulations may be sterile filtered for use as injectables or for intravenous infusion. These formulations are found to be useful for treatment of a cancerous condition in a mammalian host. Thus in another embodiment the invention is a method or use of an N-chroman or chromanol for manufacture of a medicament for treatment of a cancer in a mammalian host, characterized in that a pro-apoptotic chroman is formed as a reconstitutable dehydrated liposomal suspension precursor in combination with a liposome-forming lipid and a lyoprotectant excipient. First use of N-chroman formulations and compounds for in vivo cancer therapy in a mammalian host is demonstrated here.

### BRIEF DESCRIPTION OF THE DRAWINGS

The teachings of the present invention can be more readily understood by considering the following detailed description in conjunction with the accompanying drawings and claims, in which:

FIGS. 1A through 1D depicts molecular structures of selected N-chromans. Shown are α-tocopheryl maleate (αTOM), α-tocopheryl amide fumarate (αTAF); α-tocopheryl succinamide (αTAS), α-tocopheryl hemi-dicarboxylic acid amide (αTAX), and the functional domains I, II and III of pro-apoptogenic N-chromans.

FIG. 2 depicts the molecular structure of a chromanol, tocopheryl succinate (αTOS) with corresponding functional domains.

FIG. 3 illustrates the effect of selected chromans on mitochondrial membrane potential ΔΨ in vitro.

FIG. 4 illustrates the cytotoxic effect of selected chromans on mouse melanoma B16F10 cells in vitro.

FIG. 5 depicts the effect of liposomal αTAM on MCF-7 and B16F10 in hollow fibers surgically implanted in Balb/c mice.

FIG. 6 is a two panel figure showing side-by-side in vivo treatment with the liposomal formulations of the invention. αTOS (upper panel) and αTAM (lower panel) are administered to transgenic FVB/N c-new mice with spontaneous ductal HER2-high breast carcinomas. Tumor growth is measured over a two-week period and compared to a control group without treatment.

FIGS. 7A, 7B, and 7C represent ultrasound images of live tumors and the effect of treatment with liposomal αTAM. FIGS. 7A and 7B represent the appearance of untreated tumors on Day 1 and Day 13 respectively. FIG. 7C illustrates the appearance of the experimental tumor on Day 13, following a dose schedule of 1.5 μmol αTAM on days 0, 4, 7 and 13.

FIGS. 8A and 8B are comparative views illustrating the paradoxical effect of liposomal formulation on non-specific toxicity, here to granulocyte/macrophage colony forming cells (GM-CFC). Surprisingly, toxicity noted in in vitro studies (left panel, FIG. 8A) is not predictive of toxicity noted in vivo (right panel, FIG. 8B), where the cytotoxicity of the compounds is largely relieved by the liposomal formulation of the invention.

FIG. 9 is a plot of leukocyte counts (per uL blood) in mice control and treatment groups following administration of selected formulations of the invention. A key is provided for Week 2. The mice are studied for 3 weeks.

FIG. 10 describes the effect of varying lyoprotectant to lipid ratios on particle size and polydispersity index for a representative formulation of the invention.

FIG. 11A is a plot showing the effect of lyophilization on a liposomal particle size distribution for a representative formulation of the invention. FIG. 11B summarizes in tabular form the physical and preclinical characteristics of the liposomal products for representative two representative chromans.

FIG. 12 shows a first synthetic schema for N-chromans, and is described in more detail in Example 5.

FIG. 13 shows an alternate synthetic schema, as is useful for αTAF, for example, as described in Example 9.

FIGS. 14A and 14B tabulate properties of representative compounds and isomers. An “X” in the compound acronym indicates either ester or amide per the column headers.

### NOTATION AND NOMENCLATURE

Certain terms throughout the following description and claims are used to refer to particular features, steps or components. As one skilled in the art will appreciate, different persons may refer to the same feature, step or component by different names. This document does not intend to distinguish between components, steps or features that differ in name but not in function or action.

The drawing figures are not necessarily to scale. Certain features or components herein may be shown in somewhat schematic form and some details of conventional elements may not be shown in the interest of clarity and conciseness.

Certain meanings are defined here as intended by the inventors, ie. they are intrinsic meanings. Other words and phrases used here take their meaning as consistent with usage as would be apparent to one skilled in the relevant arts.

Tocopherol(s): Tocopherols are a family of natural and synthetic compounds containing three key structural elements, a benzopyran ring, a phenolic alcohol, and a phytyl tail. Vitamin E is an important representative of the tocopherol family. Not all tocopherols have three methyl groups on the chroman head. The simplest family member (6-hydroxy-2-methyl-2-phytylchroman) contains no methyl groups and is sometimes simply referred to as “tocol”.

All tocopherols share the phenolic alcohol as a functional group at the 6-position on the chroman head, regardless of the position of any methyl groups. In addition, the R/S stereoisomers described for the phytyl tail of α-tocopherol (3 chiral centers, 8 isomers in all) are also present in each of the other tocopherol families, e. g., beta-, delta- and gamma. Thus the total number of natural molecules, including stereoisomers, is 8×8=64.

Tocotrienol(s): Tocotrienols have structures related to the tocopherols, but possess a 3′,7′,11′-triene “tail” at the 2-position on the benzopyran ring. Again, as is the case for the tocopherols, not all tocotrienol family members have three methyl groups on the chromanol head. There are four family members (alpha-, beta-, delta-, gamma-) that are commonly encountered in food, and eight possible enantiomeric members in total, more if the desmethyl forms are considered. Tocotrienol nomenclature is not fully consistent at the level of common names. However, all natural tocotrienols share the phenolic alcohol at the 6-position on the chroman head. Adding another layer of complexity, the double bonds at the 3, 7, and 11 positions of the tail may be “cis” or “trans”, but typically are all-trans in the natural products. Tocotrienols more closely resemble ubiquinone by virtue of their polyunsaturated tail and some have been shown to have native pro-apoptotic activity.

Taken together, tocopherols, tocotrienols and their phenolic analogues and derivatives are termed “chromanols” and may be esterified at the phenolic 6-hydroxyl.

“N-chroman”, as used here, indicates a genus of chromanol analogues that are characterized by an amino substituent on a aryl carbon of the benzopyran ring. In a preferred embodiment, while not limited thereto, the amino group is substituted for the phenolic hydroxyl at the 6-position on the benzopyran ring. N-chromanyl derivatives include amides.

Chromanols and N-chromans thus include chromanol esters and N-chromanyl amides. Chromanols refer to those molecules having a phenolic hydroxyl, and ester or ether derivatives thereof. N-chromans refer to those molecules having an amine moiety on the aryl ring, and amide, imide and amino-derivatives thereof. Combinations are possible. A chromanol having a 6-hydroxyl may also have an amino group as a substituent at positions 5, 7 or 8 on the aryl ring. Taken together, chromanols and N-chromans are referred to herein as “chromans.”

The preferred members of the N-chroman and chromanol genera are characterized by dimensions conforming to a binding site for ubiquinone (coenzyme Q-10) in Complex II of the mitochondrial respiratory chain. While not bound by theory, binding of these compounds at this site is associated with generation of reactive oxygen species, free radicals that trigger apoptosis. These compounds also bind at other sites in the cell that are associated with their oncolytic and preventative properties. Bax translocation to mitochondria as well as fas and c-Jun roles in α-tocopheryl succinate induced apoptosis has also been reported.

Chromanols and N-chromans are optionally substituted at the 2-position of the benzopyran ring by T1 and T2 substituents, where T1 is a C1 to C80 hydrocarbyl, hydroxyhydrocarbyl, oxyhydrocarbyl, carboxyhydrocarbyl or phosphohydroxy hydrocarbyl, and may be saturated or unsaturated, branched or unbranched, an isoprenoid, a terpenoid, a diglyceride, a polysaccharide, or a phospholipid, for example. In the native tocopherol and tocotrienol compounds, T1 is a phytyl(4,8,12-trimethyl-tridecyl) or a trienyl(4,8,12-trimethyl-3,7,11-tridecatrienyl). The T2 substituent at the 2-position is a hydrogen, or optionally a hydrocarbyl, carbonyl, alkyl, methyl, ethyl, or carboxyl. The hydrophobic tail, which differentiates tocopherols, tocotrienols, and synthetic analogs, determines whether the molecule can bind to lipoproteins and membranes or be degraded by Phase I enzymes. The polarity of the combination of head and tail of the molecule is also relevant for its surfactant properties, its HLB, and for its overall membrane solubility, as most commonly estimated by calculating CLogP, as by the Daylight Chemical Information Systems CLogP program.

Hydrocarbyl: By“hydrocarbyl” is meant moieties containing carbon and hydrogen atoms only, with the indicated number of carbon atoms. Hydrocarbyl groups may be straight-chain or branched-chain, aliphatic or aromatic, alkanes or alkenes. Monounsaturated groups such as propene, 1-and 2-butene and propargyl, and multiple unsaturated substituents such as butadienyl, terpene, phenyl or polyphenyl, are included in this term.

“Bioisostere” refers to a chemical substituent, functional moiety, hydrophobic moiety, or signaling moiety having a chemical structure that replaces a corresponding parent chemical structure while preserving the biological properties of the parent molecular structure.

Liposome: refers to a microscopic, membranous particle or vesicle, generally rounded in shape, having one or more concentric lipid bilayers enclosing at least one aqueous compartment therein. Liposomes are formed when thin lipid films or lipid cakes are hydrated and stacks of liquid crystalline bilayers become fluid and swell. The hydrated lipid sheets detach during agitation and self-close to form large, multilamellar vesicles (LMV) which prevents interaction of water with the hydrocarbon core of the bilayer at the edges. Once these particles have formed, reducing the size of the vesicle requires energy input. Liposomes may be classified by the number of nested bilayers into multilamellar, oligolamellar, and unilamellar categories, generally in order of decreasing size and complexity of the lipid bilayer mass and are generally composed of one or more polar lipid species having a head and tail structure.

Liposomes are also defined by their method of production, which may be classified for convenience here into three categories: (1) mechanical dispersion methods, which include shaking, vortexing, sonication, high-pressure homogenization, and microfluidization; (2) dispersion by detergent-solubilization; and (3) solvent-assisted dispersion, such as ethanol injection, ether infusion, and reverse phase evaporation. Size reduction may be achieved by serial process steps, such as subjecting a crude suspension prepared by rehydration of a thin film of dried lipid to one or more high pressure extrusion or homogenization steps, and in this way suspensions having particle sizes of less than 200 nm are readily achieved. Further processing may result in particles having mean sizes in the range of 20-180 nm. A satisfactory monodisperse formulation is obtained by controlling the zeta-potential of the resulting particles. Polydispersities are typically less than 0.15, characteristics that are generally useful for injectable products.

For the manufacture of liposomes principally natural lecithins from soya beans or egg yolk or defined natural or artificial phospholipids, such as cardiolipin, sphingomyelin, lysolecithin and others are used. Phosphatidic acid may be used to increase stability in aqueous solution, if needed.

“Lecithins” refer to saturated or unsaturated phosphatidylcholine, or more informally, to egg yolk or soy phospholipids. Lecithins may be combined with phosphatidyl serine, phosphatidyl ethanolamine, phosphatidyl inositol, diphosphatidyl glycolol, phosphatidate, sphingomyelin, or the like; and further with cholesterol or electrically charged substances. Representative lecithins are derived from egg yolk, soybean. Also included are synthetic lecithins and isolated lecithins, for example, dipalmitoyl phosphatidylcholine, distearoyl phosphatidylcholine, 1-palmitoyl-2-stearoyl phosphatidylcholine, 1-stearoyl-2-palmitoyl phosphatidylcholine, hydrogenated soy phospholipids, and the like.

Ceramide, polar sterols, and more generally, cationic, anionic, and polar neutral lipid species may be used in liposome preparation. “Stealth liposomes” having prolonged circulation may also be prepared, for example by incorporating a quantity of tocopheryl polyethyleneglycol succinate (TPGS), pegylated DPPC, DSPG, or DSPE, hyaluronic acid-derivatized lipid in the product, in combination with cholesterol if needed.

Ether analogues of phospholipids, such as hexadecyl diglycerol ether, may also be used. Also of interest are the liposome-like, niosome-forming polar lipids for use as admixtures of chromans and non-ionic surfactants of the alkyl class or dialkyl polyglycerol ether class and cholesterol, with subsequent hydration in aqueous media at a temperature greater than the mean transition temperature of the surfactants. The surfactants are generally less expensive than phospholipids and may also be modified to increase targeting or AUC.

The liposomal structures of the present invention may be characterized by mixed lipid contents, where an N-chroman or chromanol is dispersed in a carrier lipid in a defined ratio. Interestingly, while spontaneous vesicular preparations of tocopheryl maleamide were associated with surprisingly severe toxicity, both localized vascular toxicity and generalized systemic toxicity upon injection, in the presence of a carrier lipid that toxicity is markedly ameliorated, for reasons not fully understood.

Other drugs or excipients may be included in a liposomal formulation. Other co-actives include camptothecins, topotecans, anthracyclines, doxorubricin, daunorubicin, etoposide, cytarabine, paclitaxel, oncolytic derivatives thereof, cisplatin, and the like. Entrapment of hydrophilic oncolytic compounds in the aqueous compartment of the liposome for example is also conceived. Thus the liposome is a drug delivery platform for co-delivery of pro-apoptotic chromans and conventional oncolytics.

“Oncolytic”: refers to a compound having the property of reversing the growth or killing a tumor cell. Certain oncolytics promote apoptosis and are termed “pro-apoptotic”.

“Bioisostere”: refers to a chemical substituent or group having physical or chemical properties that impart similar or improved biological properties when substituted in a compound having known biological properties. In drug design, the purpose of exchanging one bioisostere for another is to enhance the desired biological or physical properties of a compound without departing from the selected molecular shape. As commonly used, this definition is broadened to include substitution of functional groups that produce compounds that can sometimes have similar biological activities.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, particular features, structures, or characteristics of the invention may be combined in any suitable manner in one or more embodiments.

“Conventional”—refers to a term or method designating that which is known and commonly understood in the technology to which this invention relates.

Unless the context requires otherwise, throughout the specification and claims which follow, the word “comprise” and variations thereof, such as, “comprises” and “comprising” are to be construed in an open, inclusive sense, that is as “including, but not limited to”.

The appended claims are not to be interpreted as including means-plus-function limitations, unless such a limitation is explicitly recited in a given claim using the phrase “means for”, and are not to be interpreted as Markush claims unless explicitly recited with the language, “selected from the group consisting of”.

### DETAILED DESCRIPTION

Although the following detailed description contains specific embodiments and details for the purposes of illustration, one of skill in the art will appreciate that many variations, combinations, substitutions and modifications of the following details are within the scope of the invention. Accordingly, the exemplary embodiments and details of the invention described below are set forth without any loss of generality to, and without imposing limitations upon, the claimed invention.

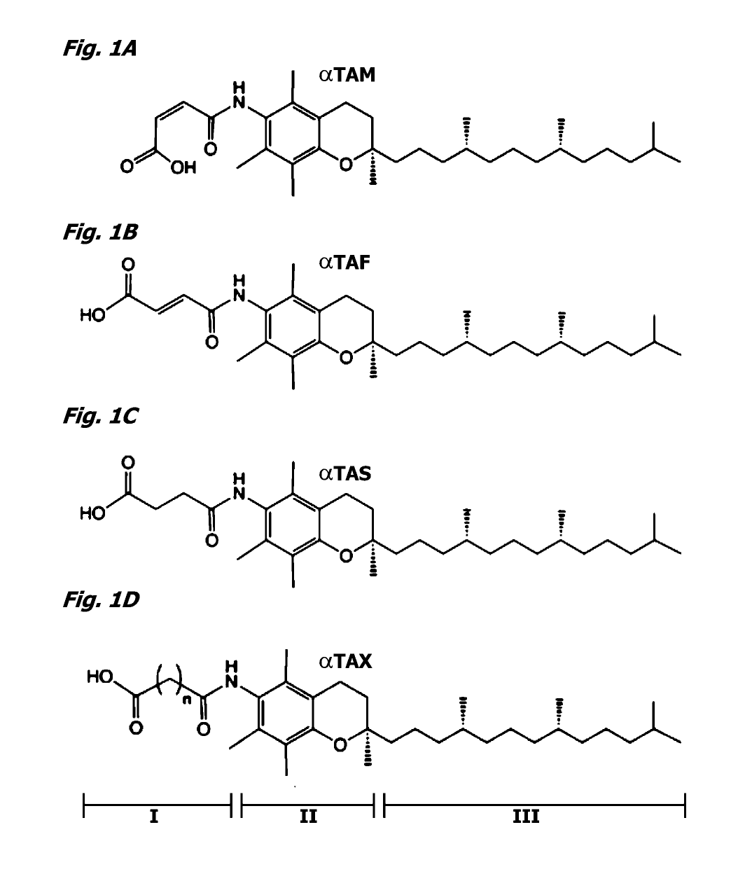

Turning now to the figures, FIGS. 1A through 1D depicts molecular structures of selected N-chromans. Shown are α-tocopheryl maleate (αTOM), α-tocopheryl amide fumarate (αTAF); α-tocopheryl succinamide (αTAS), and α-tocopheryl hemi-dicarboxylic acid amide (αTAX).

As illustrated in FIG. 1, N-chromans have a molecular architecture characterized by an amino substituent on the chroman head. This amino substituent permits facile formation of amide derivatives that are redox silent and pro-apoptotic when provided with a terminal carboxyl of the appropriate molecular dimensions. In a preferred embodiment of the invention, the liposomal formulations of the invention may be used to deliver members of the N-chroman genus to a mammalian host in need thereof.

N-chromanyl amides may include N-chromanyl amino hemi-dicarboxylic acid amides, where the hemi-dicarboxylic acid is an unsaturated dicarboxylic acid residue having 0 to 5 carbons, a saturated dicarboxylic acid residue having 0 to 5 carbons, an oxalate, a malonate, a maleate, a malate, a fumarate, a butenedioic acid, a glutarate, a pentenedioic acid, a phthalic acid, or a terephthalic acid, while not limited thereto.

Preferred members of the N-chroman genus include hemi-carboxylic acids of tocopheryl oxalamide, tocopheryl malonamide, tocopheryl maleamide, tocopheryl fumaramide, tocopheryl succinamide, tocopheryl malamide (hemi-carboxylic acid of malic acid amide), tocopheryl glutaramide (hemi-carboxylic acid of glutaric acid amide), tocopheryl phthalamide (hemi-carboxylic acid of phthalic acid amide) and its isomers, and also include propanedioic acid amides, butenedioic acid amides, and pentanedioic acid amides and their saturated, unsaturated, and hydroxylated isomers, and their stereoisomers more generally.

N-chromans are generally characterized by a lower IC₅₀ against cancer cell lines and may have a higher systemic toxicity when administered in solutions in ethanol or DMSO or as self-vesiculated vesicles.

These compounds are representative of a genus of pro-apoptotic N-chromans, which, referring to FIG. 1D, includes compounds having a chroman head (II), a hydrophobic tail domain (III), and a pro-apoptotic, “redox silent” substituent (I) on the chroman head. These domains of the molecules are termed here the “hydrophobic moiety” (III), the “signaling moiety” (II) and the “functional moiety” (I).

The hydrophobic moiety (III) is fundamentally a docking domain responsible for lipoprotein binding and for membrane binding and penetration, but may also be associated with some apoptotic activity, as with synthetically modified tocotrienols. The signaling moiety (II) modulates certain signaling pathways such as the protein phosphate 2/protein kinase C pathway and is characterized by a pattern of methylation that confers different levels of activity for the alpha, gamma and delta isomers of tocopherol and tocotrienol analogues. The signaling moiety corresponds to what has traditionally been termed the “chroman head” in analyzing tocopherol-like molecules and their analogues. In native tocopherols and tocotrienols, the phenolic hydroxyl and benzopyran ring of the chroman head is a potent antioxidant. However, the functional moiety (I) has proven key to much higher levels of apoptotic potency by blocking the antioxidant potential of the phenolic hydroxyl when present. Tocopheramine, in which the phenolic hydroxyl is substituted by an amine function, is redox silent and may be derivatized to form pro-apoptotic N-chomans.

All three domains are needed for inducing apoptosis. The details of these domains are still being explored by a painstaking process of synthesis and biological testing.

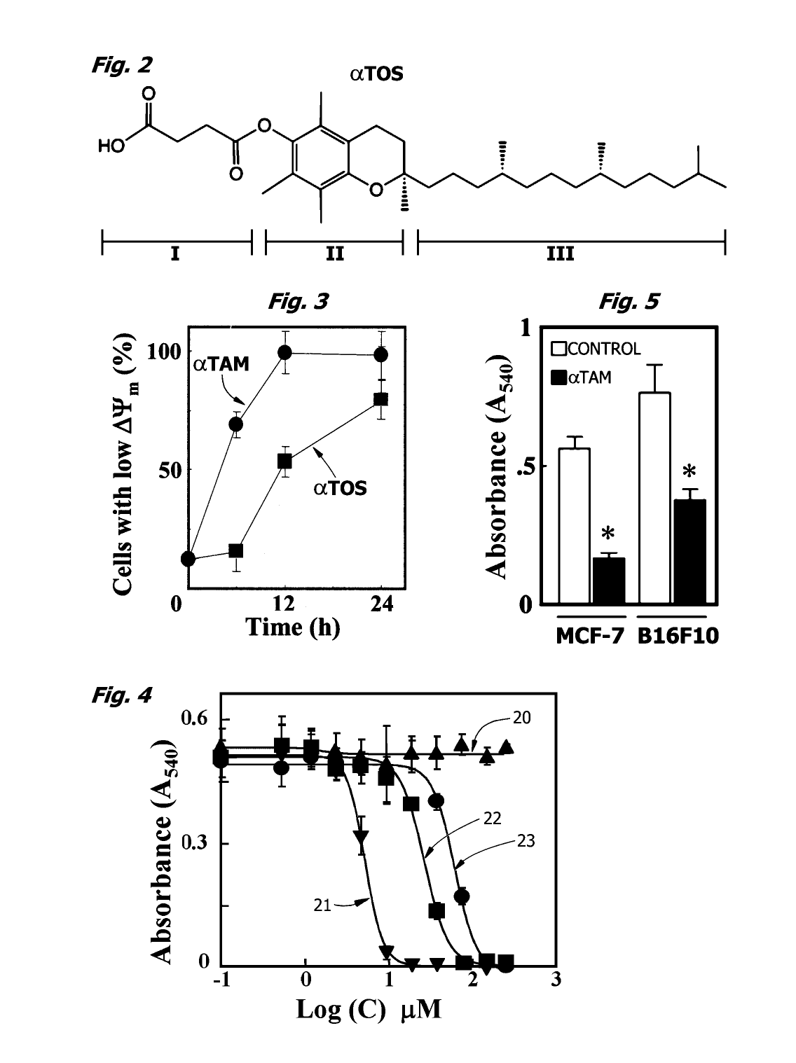

The most studied functional moiety analogues are tocopheryl monoesters of dicarboxylic acids. FIG. 2 depicts the molecular structure of a dicarboxylic acid ester, tocopheryl succinate (αTOS), a representative chromanol. These esters bear a terminal acidic carboxyl and have dimensions and binding characteristics that favor binding to the Coenzyme Q10 binding site of Complex II (succinate-ubiquinone binding site) of the mitochondrial respiratory chain. α-Tocopherol maleate ester (αTOM) and α-tocopheryl fumarate ester (αTOF) have also been shown to have yet higher apoptotic potentials, suggesting that an optimal candidate may yet be found and be administered by the liposomal formulations of the invention.

Domains I, II and III are shared by pro-apoptogenic chromanols and N-chromans (see Wang et al, 2006, Vitamin E analogues as anticancer agents, Mol Nutr Food Res 50:675-685; Neuzil et al, 2007, Vitamin E analogs, a novel group of “mitocans” as anticancer agents. Mol Pharm 71:1185-99; Yu et al, 1999, Induction of apoptosis in human breast cancer cells by tocopherols and tocotrienols, Nutr Cancer 33:26-32; Birringer et al, 2003, Vitamin E analogues as inducers of apoptosis: structure-function relation, Brit J Cancer 88:1948-55; and Tomic-Vatic et al, 2005, Vitamin E amides, a new class of vitamin E analogues with enhanced proapoptotic activity, Intl J Cancer 117:188-93).

Together, N-chromans and chromanols are termed “chromans”, and may be configured to be pro-apoptotic by derivatization through a process of trial and error as has been described here.

FIG. 3 illustrates the effect of selected chromans on mitochondrial membrane potential ΔΨ in vitro. It is shown that vitamin E amides such as αTAM and αTAS are potent in dissipating mitochondrial inner transmembrane potential in Jurkat T cells. Deviations in ΔΨ were estimated by flow cytometry followed by cell staining with the polychromatic JC-1 probe.

FIG. 4 illustrates the cytotoxic effect of selected chromans on mouse melanoma B16F10 cells in vitro. The murine melanoma cell line B16F10 was used for testing the effects of αTOH (20) and its analogues αTOS (23), αTOM (22) and αTAM (21). The cytotoxic potential of the four agents is presented with the order of αTAM>αTOM>αTOS>>αTOH. No toxicity was observed for the redox-active αTOH (20). Similarly, the IC₅₀ values followed the same trend in multiple cell lines. In human breast cancer cell line MCF-7, IC₅₀s were 13.3 μM for αTAM, 35.5 μM for αTOM, and 63.5 μM for αTOS. IC₅₀ values were calculated from MTT activity (Bank, U et al, 1991, Measurement of cellular activity by means of the MTT-test. Allerg Immunol 37:119-123).

FIG. 5 depicts the effect of liposomal αTAM on MCF-7 and B16F10 in vivo. Hollow fibers were first seeded with MCF-7 or B16F10 cells and implanted surgically in the peritoneum of Balb/c mice. The mice were then injected at 48 hour intervals with 3 doses of liposomal αTAM at 25 mg/kg each; the hollow fibers were then removed and proliferation of the cells evaluated using the MTT assay. Liposomal chromans inhibit proliferation of cancer cells and suppress breast carcinomas.

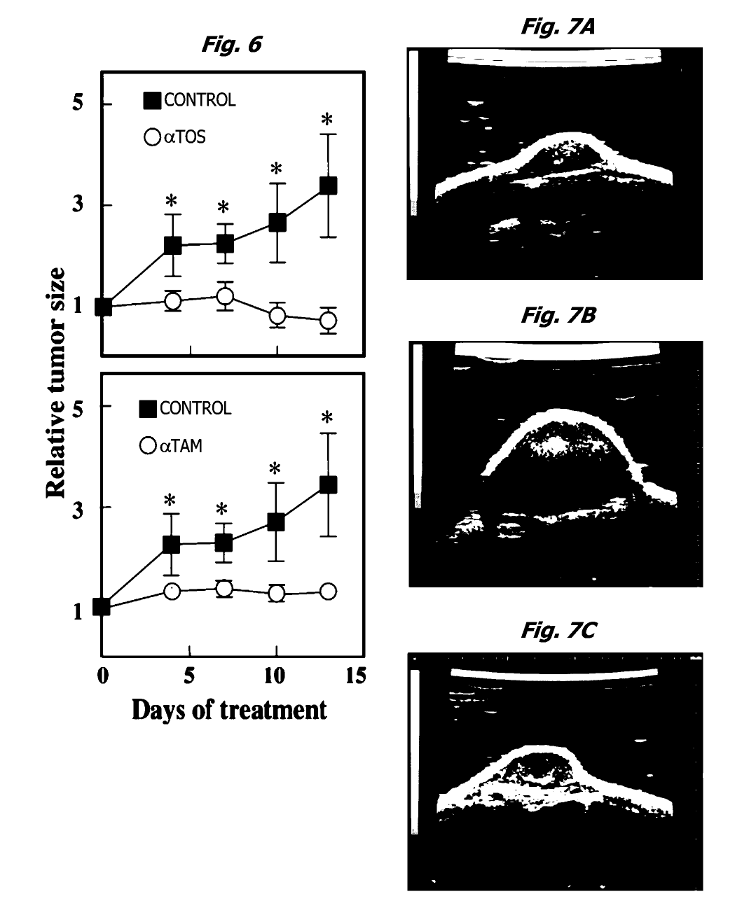

FIG. 6 is a two panel figure showing side-by-side in vivo treatment with the liposomal formulations of the invention. αTOS (upper panel) and αTAM (lower panel) are administered to transgenic FVB/N c-neu mice with spontaneous ductal HER2-high breast carcinomas. Tumor growth is measured over a two-week period and compared to a control group without treatment. Liposomal preparations of both αTOS and αTAM suppressed breast carcinomas in the c-neu mice by 90-100% when intravenously injected in amounts corresponding to approximately 15 umol αTOS and 1.5 umol αTAM per dose, a ten-fold difference in dosage. Injections were given on days 0, 4, 7 and 13. Importantly, neither of the two compounds was toxic to the c-neu mice when formulated with a liposomal formulation of the invention. Happily so, this was especially surprising and encouraging in the case of αTAM, which is extremely toxic when administered as solution in DMSO, and represents a first actual use of an N-chroman in oncotherapy of a mammalian host in need thereof.

Related observations suggest that αTAM doses of 500 μg per 20 g mouse were tolerated when given in liposomal formulation as described. This corresponds to a safe dose of 25 mg/kg, approximately six times the therapeutic dose (estimated in humans to be around 100-200 mg/m²), and would seem to indicate that doses of 1.75 g αTAM are feasible for human use. However, higher doses were not studied here and a maximal tolerated dose has not yet been established.

FIGS. 7A, 7B, and 7C represent ultrasound images of live tumors and the effect of treatment with a liposomal αTAM formulation of the invention. FIGS. 7A and 7B represent the appearance and growth of untreated tumors on Day 1 and Day 13 respectively. FIG. 7C illustrates the size of the experimentally treated tumor on Day 13, following a dose schedule of 1.5 μmol αTAM/animal on days 0, 4, 7 and 13.

The general toxicity of liposomal preparations of αTAM was studied further in mice by the Berlin Test. Untreated mice and mice treated with empty liposomes were used as controls. None of the typical symptoms of toxicity, including motor disorder, respiratory problems, apathy, horrent fur, behavioral changes, anorexia and loss of body mass, were observed immediately after application of the N-chroman within the subsequent 10 d in either Balb/c or C57B1 mice when administered in a liposome formulation of the invention. No morphological changes of inner organs were observed after dissection and microscopic examination. Contrastingly, free αTAM administered in DMSO at 5 mg/kg caused rapid death of the animals. Symptoms including spasms and heavy breathing pointed to neurotoxicity, embolie or anaphylactic reaction. Therefore, we have discontinued experiments that involved the use of non-liposomal formulations of αTAM.

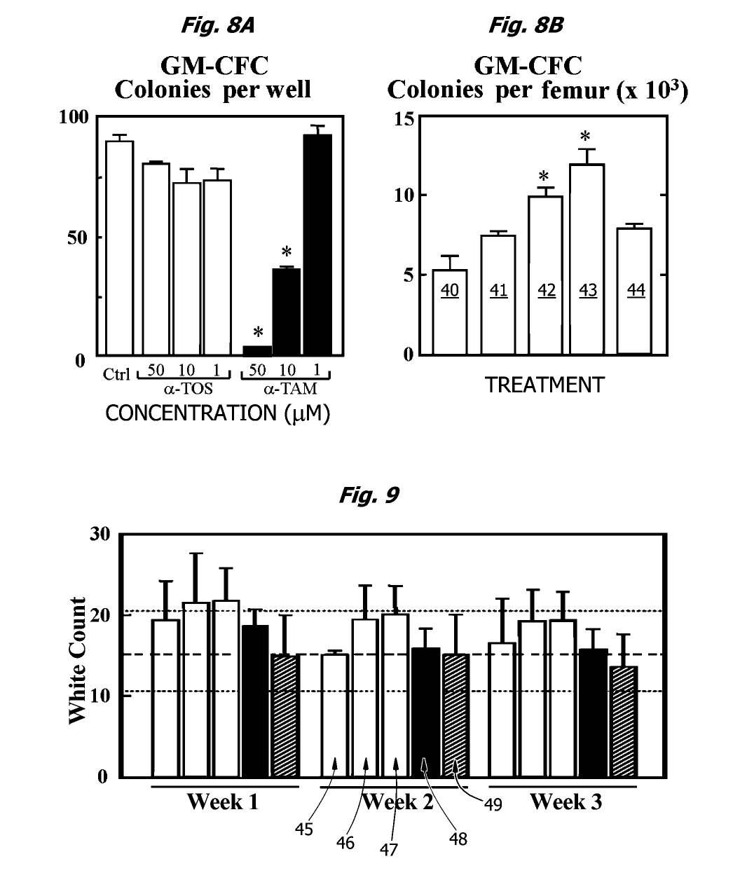

FIGS. 8A and 8B are comparative views illustrating the paradoxical effect of the liposomal formulation on non-specific toxicity, here to granulocyte/macrophage colony forming cells (GM-CFC). Surprisingly, toxicity noted in in vitro studies (left panel, FIG. 8A) is not predictive of toxicity noted in vivo (right panel, FIG. 8B), where the cytotoxicity of the compounds is largely relieved by the liposomal formulation of the invention. In the left panel, GM progenitors were isolated from Balb/c mouse bone morrow and treated with αTOS (left) or αTAM (right, black bars) at the concentrations shown, the number of colonies was counted and expressed per individual well. In the right panel, Balb/c mice were injected i.p. with the vehicle (40), empty liposomes (41) liposomal αTOS at 100 mg/kg (42), αTAM at 10 mg/kg (43), or αTAM at 25 mg/kg (44), and GM-CFC colonies in femoral bone marrow counted 48 h later (* denotes statistically significant differences with pb0.05 in comparison to control). Thus, toxicity against precursor/haematopoetic stem cells in vivo is not induced by administration of liposomal αTAM, although the compound is toxic in vitro. On the contrary, in vivo application of both liposomal formulations resulted in a 1.5 to 2.5-fold stimulation of bone marrow proliferation reflected by an increase in GM-CFC progenitors after administration! This surprising result is paradoxical, and points to the importance of the formulation vehicle as a technological advance in achieving clinical trials of these compounds.

To reiterate, apparent stimulation of haemopoetic precursors in bone marrow is noted (FIG. 8B) with both αTOS and αTAM at intermediate doses. While no explanation is forthcoming at this time, the data is encouraging, as most cytolytics used in cancer therapy are associated with immunosuppression and leucopenia. Uses for vitamin E analogues in combination with immunostimulant cancer therapies have previously been noted for αTOS but not for αTAM formulations. αTOS is a hydrolysable prodrug of Vitamin E; αTAM is not.

FIG. 9 is a plot of leukocyte counts (per μL blood) in mice control and treatment groups following administration of selected formulations of the invention. A key is provided for Week 2. Balb/c mice were injected IV with the vehicle (45), with empty liposomes (46), with liposomal αTOS (47), with liposomal αTAM at 10 mg/kg (48, black bar), or with liposomal αTAM at 25 mg/kg (49, hatched bar). The mice were studied for 3 weeks by drawing 20 μL of blood from each animal and counting the number of leukocytes. The dashed horizontal line shows the normal leukocyte count in healthy untreated animals, with standard deviation indicated by the dotted lines. No significant differences between treated and control groups were found. Thus there is a good correlation between the in vivo effects of liposome preparations of chromans on bone marrow proliferation (FIG. 8) and leukocyte count in peripheral blood after intravenous administration of the formulations.

### Examples

### Example 1

### Prior Art I

In earlier work to develop a formulation of αTAM for preclinical study, the compound was first dissolved in DMSO or ethanol as has been characteristic of studies of this kind (Weber et al, 2002, Vitamin E succinate is a potent novel antineoplastic agent with high selectivity and cooperativity with tumor necrosis factor-related apoptosis-inducing ligand (Apo2 ligand) in vivo, Clin Cancer Res 8:863-69; Yu et al, 1999, Induction of apoptosis in human breast cancer cells by tocopherols and tocotrienols, Nutr Cancer 33:26-32). Unfortunately, formulations of αTAM of this kind were routinely found to be extremely toxic. One or two injections of αTAM in DMSO in Balb/c, C57B1 or FVB/N c-neu mice caused death of the animals, associated with severe neurotoxicity and anaphylactic shock.

### Example 2

### Prior Art II

Taking advantage of the known self-vesiculating property of polar tocopherol analogues (Kogure et al, 2003, Potentiation of anti-cancer effect by intravenous administration of vesiculated α-tocopheryl hemisuccinate on mouse melanoma in vivo, Cancer Lett 192:19-24; Ramanathapuram et al, 2005, Chemo-immunotherapy of breast cancer using vesiculated α-tocopheryl succinate in combination with dendritic cell vaccination, Nutr Cancer 53:177-193), vesicles of αTAM were prepared by rehydrating lipid films with agitation and reduction of particle size. Immediately following tail vein injection, mice treated with these formulations became irritable, experienced tremors, and at least one mouse died. Subsequently, gangrene in the tail vein injection site was noted, discouraging further preclinical work in this direction.

### Example 3

### Preparation of Chromans in Lyophilized Liposomal Suspensions

In a modification of procedures for preparing liposomes (Turanek, J et al, 2003, Preparation of sterile liposomes by proliposome-liposome method, Methods Enzymol 367: 111-125), N-chroman:phosphatidylcholine mixed liposomes or chromanol mixed liposomes were prepared in a single stage process using the proliposome-liposome method, or hydration of a lipid film, followed by extrusion through polycarbonate filters with different pore size (Turanek, J et al, 2003, Preparation of sterile liposomes by proliposome-liposome method, Methods Enzymol 367: 111-125). Liposome preparations containing αTAM and αTOS have been prepared.

For αTAM liposomes, briefly, a mixture of EPC (egg phosphatidylcholine, 99%; Avanti Polar Lipids, Alabaster, Ala.) and αTAM was dissolved in chloroform and transferred into a round-bottom flask. For initial studies, lipid/αTAM in molar ratios of 95:5; 90:10; 85:15 and 80:20 may be used. The organic solvent was removed using a rotary vacuum evaporator (Laborota 4000, Heidolph, Del.) yielding a dry thin lipid film (40° C., 4 h). The lipid film was then hydrated with an aqueous phase (20 mM HEPES buffer, pH 7.20, 0.2 mm filtered) and converted to the suspension of MLVs (lipid concentration of 10 mg/mL) by continuous shaking (30 min).

In the case of saturated PC (for example dipalmitoyl phosphatidylcholine) the temperature of the hydrating medium should be above the gel-liquid crystal transition temperature (T_{c} or Tₘ) of the lipid with the highest T_{c} before adding to the dry lipid. After addition of the hydrating medium, the lipid suspension should be maintained above the T_{c} during the hydration period. Liposomes are not generated if the procedures are carried out below the gelphase/liquid crystal-phase transition temperature of the lipid used. For high transition temperature lipids, this is easily accomplished by transferring the lipid suspension to a round bottom flask and placing the flask on a rotary evaporation system without a vacuum. Spinning the round bottom flask in a warm water bath maintained at a temperature above the T_{c} of the lipid suspension allows the lipid to hydrate in its fluid phase with adequate agitation. No oversaturation or precipitation was noted.

The conventional intermediate freezing and thawing step (see for example U.S. Pat. No. 4,880,635 “Vesicle Preparation” to Janoff, and references therein) for liposome preparation was omitted in order to preserve the oligolamellar morphology of the liposomes with a low internal volume of the water phase. Oligolamellar characteristics and low internal aqueous volume were selected in order to minimize toxicity and stability of the formulation. MLVs were then sequentially extruded seven times through polycarbonate filters (Whatman, Kent UK) of various pore sizes (400, 200, 100, 80 and 50 nm) at room temperature to find the optimal extrusion procedure yielding a size fraction appropriate for intravenous administration.

A hand-operated mini-extruder (Avanti Polar Lipids) was used for preparation of small volumes of liposomes (up to 1 ml). Large-volume liposomes were extruded using a high-pressure cell attached to the FPLC instrument (GE Healthcare UK) (Turanek, J, 1994, Fast-protein liquid-chromatography system as a tool for liposome preparation by the extrusion procedure, Anal Biochem 218, 352-357). Preferably, the aqueous compartment in the product, prior to lyophilization, is less than 2% of the original water in the preparation, and essentially no water remains after lyophilization.

Size and zeta-potential measurements, DLS (dynamic light scattering) and micro-electrophoresis were performed using a NanoSizer SZ (Malvern, Worcestershire UK) to measure the size and zeta-potential of liposome preparations, using phospholipids at 1 mg/ml in PBS and temperature of 25° C. Disposable cells were used for zeta-potential measurements.

Mean size distributions may be controlled within a window of 20 to 180 nm, more preferably 50 to 140 nm for MLV, as are suitable for terminal sterile filtration. In vivo testing was performed with lots having a size range between 120 and 140 nm, but the size may be reduced by further processing. Size distributions were generally monomodal and consisted of MLVs. Zeta potentials were generally in the range of −10 to −15 mV and were monomodal. Polydispersities were in the range of 0.1 to 0.14; polydispersities in the range of 0.05 to 0.2 are generally acceptable for intravenous, injectable, or instillation use.

For preparation of lyophilized liposomal solids, the extruded liposomes were mixed with the appropriate amount of sucrose or other lyoprotectant and sterilized by filtration through 0.22-mm filters (Millex-MP Filter Unit; Millipore, Billerica, Mass.). Lipid/sucrose molar ratios were 1:1, 1:3, 1:5, 1:7 and 1:10 were selected for investigation. Aliquots of the liposomal preparation (10 mg/mL of total lipid content) were filled into 20-mL sterile vials. These vials with 1.5 mL of liposomes were frozen at −80° C. in a freezer and then lyophilized using the Lyovac GT2 instrument (GEA Lyophil GmbH, Hurth Del.). The samples were placed into the drying chamber precooled to −45° C. The lyophilization procedure was run for 24 h at 8 Pa. After this period, a second drying step was applied at 25° C. for 12 h under 20 Pa. Preferably the lyophilized preparations were sealed under argon atmosphere to displace residual oxygen, thereby reducing lipid oxidation and formation of lipid hydroperoxides. The lyophilized samples were stored at 2-8° C. for further characterization and stability studies.

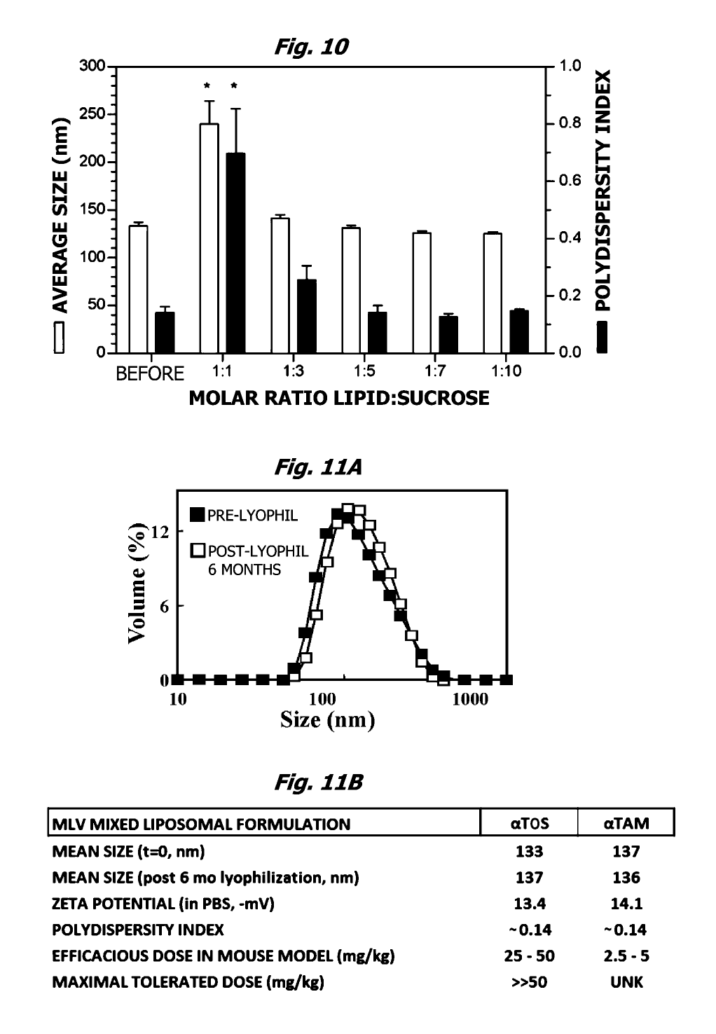

FIG. 10 describes the effect of varying lyoprotectant-to-lipid ratios on particle size and polydispersity index for a representative formulation of the invention. The lipid:sucrose molar ratio of 1:5 was found sufficient to preserve the physical (size, polydispersity) and chemical (content of αTAM or αTOS) properties of the liposomal preparations.

A variety of lyoprotectants in addition to sucrose or trehalose may be used. These include arabinose, erythritol, fructose, galactose, glucose, lactose, maltitol, maltose, maltotriose, mannitol, mannobiose, mannose, ribose, sorbitol, saccharose, xylitol, xylose, dextran, or mixtures thereof. A plasticizer may also be used to control the melting temperature of the amorphous glass phase. Plasticizers include glycerol, dimethylsulfoxide, lower molecular weight polyethylene glycol, propylene glycol, diethylene glycol dimethylether, triethyleneglycol dimethyl ether, tetraethylene glycol dimethyl ether, N,N-dimethylacetamide, N,N-dimethylformamide, tetramethyurea, water, or mixtures thereof. Plasticizers can also be useful in controlling the degree of crystallinity of the solid phase lyophil.

Binders are also used to control rehydration kinetics and for powder handling. Binders include polyvinylpyrrolidinone, high molecular weight polyethylene glycol, a block copolymer of polypropyleneglycol and polyethylene glycol, polyacrylate, polymethylmethacrylate, poly-(d-1-lactide-co-glycolide), triethylene glycol dimethylether, butyl diglyme, chitosan, a cellulose, a methylcellulose, an alginate, an albumin, a dextran, a starch, or a gelatin. is polyvinylpyrrolidinone, high molecular weight polyethylene glycol, a block copolymer of polypropyleneglycol and polyethylene glycol, polyacrylate, polymethylmethacrylate, poly-(d-1-lactide-co-glycolide), triethylene glycol dimethylether, butyl diglyme, chitosan, a cellulose, a methylcellulose, an alginate, an albumin, a dextran, a starch, a gelatin, or mixtures thereof.

FIG. 11A is a plot showing the effect of lyophilization on a liposomal particle size distribution for a representative formulation of the invention. A size distribution for the raw formulation was obtained by dynamic laser scattering; the formulation was then lyophilized, reconstituted and reexamined after 6 month storage under refrigeration. Little or no growth of particle size by volume was noted, indicating a degree of physical stability equivalent to that expected of clinical lots of a drug. Also, there was virtually no difference in the content of αTAM and αTOS (as assessed by HPLC) in the lyophilized liposomal samples. Samples sealed under argon have been stored for up to 12 months at 4° C. (data not shown) with no change in physical and chemical properties. The products are thus also superior to emulsion technologies in stability.

FIG. 11B summarizes physical and biological data collected for the liposomal formulations of Example 3. Comparing an αTOS and an αTAM formulations, mean sizes are seen to be generally stable after 6 months of refrigerated storage, and are likely to be stable for longer periods under these conditions. The Zeta potential of an αTOS formulation was −13.4 mV, and comparable to αTAM, where −14.1 mV was measured. Polydispersity index was essentially equivalent between replicate preparations. However, a ten-fold lower efficacious dose for αTAM was obtained in a whole animal model in transgenic FVB/N c-neu mice with spontaneous ductal HER2-high breast carcinomas (FIGS. 6,7). Because of limited compound availability, maximal tolerated dose (MTD) could not be estimated, but is greater than 50 mg/kg for αTOS. MTD for the N-chroman is not known. The higher efficacy of αTAM formulations is due to a number of factors, the active compound has been shown to have increased pro-apoptotic potency (FIGS. 3-5), to resist enzymatic hydrolysis, but may also relate to the higher AUC obtained with liposomal formulations and reduced non-specific toxicity also attributed to the formulation.

### Example 4

### Liposomes Containing αTAM and Paclitaxel

In one experiment, we encapsulated paclitaxel in an αTAM-containing liposome formulation of the invention. No instability or incompatibility was noted, indicating that co-oncotherapeutics containing dual actives are readily formulated.

### Example 5

### Preparation of αTAM

Tocopheramine may be synthesized as described elsewhere (Mandavian et al 2009, a novel synthesis of tocopheryl amines and amides, Tetrahedron Lett 50:19-21; Mazzini et al, 2009, Efficient synthesis of vitamin E amines, Eur J Org Chem 2009: 2063-68).

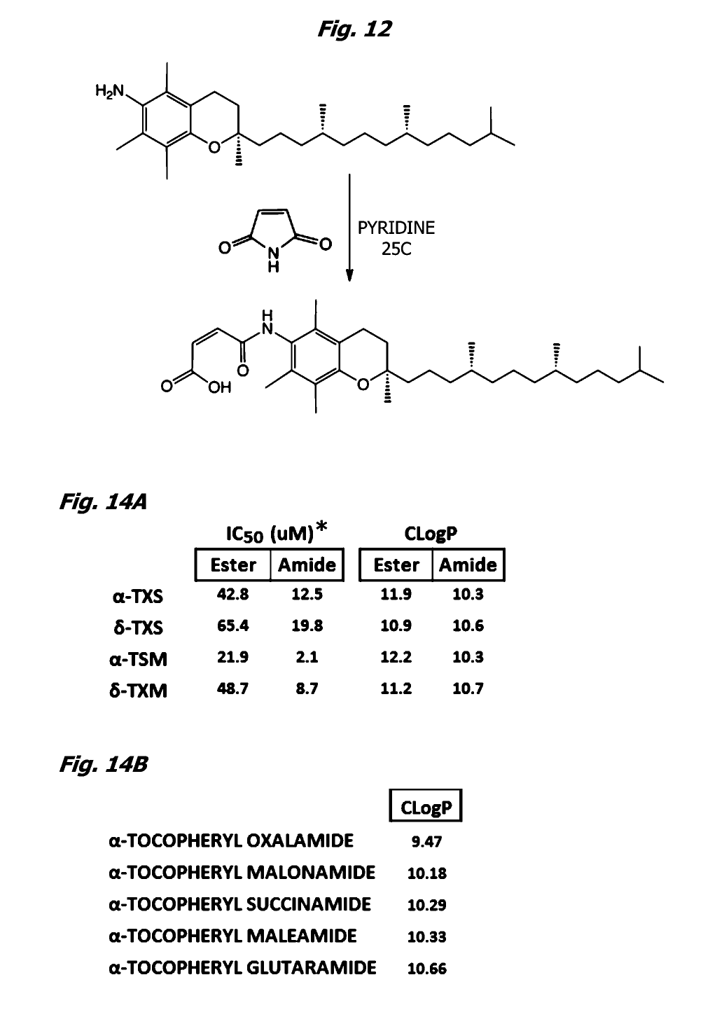

Following the schematic of FIG. 12, α-tocopheramine (725 mg, 1.69 mmol) was added to anhydrous dichloromethane (10 mL), and anhydrous pyridine (1.5 ml) in a round bottom flask. Then maleic anhydride (625 mg, 6.37 mmol) was added and the reaction mixture was stirred overnight at room temperature. During this time, the reaction changed color from yellow to dark brown. The progress of the reaction was monitored by TLC (5:1 hexane/EtOAc). Upon completion, methanol (2 ml) was added and the volatile solvents were removed on the rotavap with mild heating. Then the residual pyridine was removed on the high vacuum pump overnight. The crude was purified using flash chromatography with gradient solvent mixture 4:1:0.6% toluene/EtOAc/AçOH to 3:1:0.6% toluene/EtOAc/AcOH). Pure product (820 mg, 92%) was a yellow solid.

αTAM: yellow solid; ¹H NMR (CDCl₃, 300 MHz) δ 0.853 (d, J=6.4 Hz, 3H), 0.871 (d, J=6.5 Hz, 3H), 0.904 (d, J=6.5 Hz, 6H), 1.25 (s, 3H), 1.05-1.58 (m, 18H), 1.48-1.59 (m, 3H), 1.74-1.83 (m, 2H), 2.05 (s, 3H), 2.07 (s, 3H), 2.11 (s, 3H), 2.59-2.64 (t, J=6.8 Hz, 2H), 6.47 (d, J=12.7 Hz, 1H), 6.64 (d, J=12.7 Hz, 1H); ¹³C NMR (CDCl₃, 300 MHz) δ 12.24, 14.35, 15.17, 19.89, 19.98, 21.00, 21.23, 22.79, 22.93, 24.11, 24.66, 24.98, 26.42, 28.21, 29.82, 31.16, 32.12, 32.92, 33.01, 37.49, 37.64, 39.57, 75.75, 117.77, 123.36, 124.00, 127.52, 128.79, 152.82, 131.64, 137.50, 165.30, 168.90; ESIMS m/z=526.3902 (M⁺-1).

A first use of a liposomal formulation of α-tocopheryl maleamide in phosphatidylcholine for in vivo oncolysis was then demonstrated. α-Tocopheryl maleamide represents a novel class comprising apoptogenic N-chromans with a non-cleavable amide bond, endowing the class with higher pro-apoptotic effects in vitro than earlier studied chromanols (Tomic-Vatic et al, 2005, Vitamin E amides, a new class of vitamin E analogues with enhanced proapoptotic activity, Intl J Cancer 117:188-93). Other members of the genus of α-tocopheryl hemi-dicarboxylic acid amides are described in the following examples.

### Example 6

### Preparation of δTAM

To a round bottom flask was added δ-tocopheramine (1.7 mmol), anhydrous dichloromethane (10 mL), and anhydrous pyridine (1.5 ml). Then maleic anhydride was added in excess and the reaction mixture was stirred overnight at room temperature. The progress of the reaction was monitored by TLC (5:1 hexane/EtOAc). Upon completion, methanol (2 ml) was added and the volatile solvents were removed on the rotavap with mild heating. Residual pyridine was removed on high vacuum overnight. The crude product was purified using flash chromatography with gradient solvent mixture 4:1:0.6% toluene/EtOAc/AçOH to 3:1:0.6% toluene/EtOAc/AcOH).

δTAM: yellow solid; ¹H NMR (CDCl₃, 300 MHz) δ 0.863 (d, J=6.7 Hz, 3H), 0.877 (d, J=6.6 Hz, 3H), 0.885 (d, J=6.5 Hz, 6H), 1.28 (s, 3H), 1.02-1.42 (m, 18H), 1.49-1.58 (m, 3H), 1.73-1.85 (m, 2H), 2.17 (s, 3H), 2.74-2.78 (m, 2H), 6.93 (d, J=12.9 Hz, 1H), 6.77 (d, J=13.0 Hz, 1H), 7.23 (s, 1H), 7.31 (s, 1H); ¹³C NMR (CDCl₃, 300 MHz) δ 16.29, 16.37, 19.83, 19.95, 21.16, 22.63, 22.83, 22.93, 24.34, 24.64, 24.99, 28.17, 29.90, 31.21, 32.88, 32.98, 37.46, 37.62, 39.55, 40.34, 76.70, 120.25, 121.13, 121.92, 127.30, 127.56, 133.76, 135.59, 151.03, 164.09, 167.02; ESIMS m/z=498.3589 (M⁺-1).

### Example 7

### Preparation of αTAS

To a round bottom flask was added α-tocopheramine (1.7 mmol), anhydrous dichloromethane (10 mL), and anhydrous pyridine (1.5 ml). Then succinic anhydride was added in excess and the reaction mixture was stirred overnight at room temperature. The progress of the reaction was monitored by TLC (5:1 hexane/EtOAc). Upon completion, methanol (2 ml) was added and the volatile solvents were removed on the rotavap with mild heating. Residual pyridine was removed on high vacuum overnight. The crude product was purified using flash chromatography with gradient solvent mixture 4:1:0.6% toluene/EtOAc/AçOH to 3:1:0.6% toluene/EtOAc/AcOH).

αTAS: yellow solid; ¹H NMR (CDCl₃, 300 MHz) δ0.864 (d, J=6.4 Hz, 3H), 0.874 (d, J=6.4 Hz, 3H), 0.922 (d, J=6.5 Hz, 6H), 1.25 (s, 3H), 1.05-1.42 (m, 18H), 1.49-1.58 (m, 3H), 1.73-1.85 (m, 2H), 2.05 (s, 3H), 2.09 (s, 3H), 2.12 (s, 3H), 2.57-2.63 (m, 2H), 2.71-2.76 (t, J=6.6 Hz, 2H), 2.81-2.85 (t, J=6.6 Hz, 2H); ¹³C NMR (CDCl₃, 300 MHz) δ 12.20, 14.16, 15.00, 19.84, 19.96, 21.01, 21.25, 22.84, 22.93, 24.06, 24.66, 25.01, 28.18, 30.17, 30.88, 31.34, 32.93, 33.00, 37.49, 37.66, 39.57, 40.34, 75.66, 117.91, 122.94, 123.51, 125.07, 125.60, 151.40, 131.40, 133.43, 171.67, 177.23; ESIMS m/z=528.4058 (M⁺-1).

### Example 8

### Preparation of δTAM

To a round bottom flask was added δ-tocopheramine (1.7 mmol), anhydrous dichloromethane (10 mL), and anhydrous pyridine (1.5 ml). Then succinic anhydride was added in excess and the reaction mixture was stirred overnight at room temperature. The progress of the reaction was monitored by TLC (5:1 hexane/EtOAc). Upon completion, methanol (2 ml) was added and the volatile solvents were removed on the rotavap with mild heating. Residual pyridine was removed on high vacuum overnight. The crude product was purified using flash chromatography with gradient solvent mixture 4:1:0.6% toluene/EtOAc/AçOH to 3:1:0.6% toluene/EtOAc/AcOH).

δTAS: yellow solid; ¹H NMR (CDC1₃, 300 MHz) 0.834 (d, J=6.6 Hz, 3H), 0.855 (d, J=6.5 Hz, 3H), 0.860 (d, J=6.5 Hz, 6H), 1.23 (s, 3H), 1.03-1.40 (m, 18H), 1.47-1.55 (m, 3H), 1.69-1.84 (m, 2H), 2.12 (s, 3H), 2.56-2.83 (m, 6H), 6.93 (s, 1H), 7.11 (s, 1H); ¹³C NMR (CDCl₃, 300 MHz) δ 16.27, 17.84, 19.84, 19.96, 21.17, 22.63, 22.84, 22.93, 24.34, 24.65, 25.01, 28.18, 29.91, 31.05, 31.35, 31.78, 32.90, 33.00, 37.48, 37.64, 39.57, 40.24, 76.33, 119.40, 120.83, 121.17, 126.83, 128.34, 149.48, 170.20, 175.80; ESIMS m/z=500.3745 (M⁺-1).

### Example 9

### Preparation of αTAF

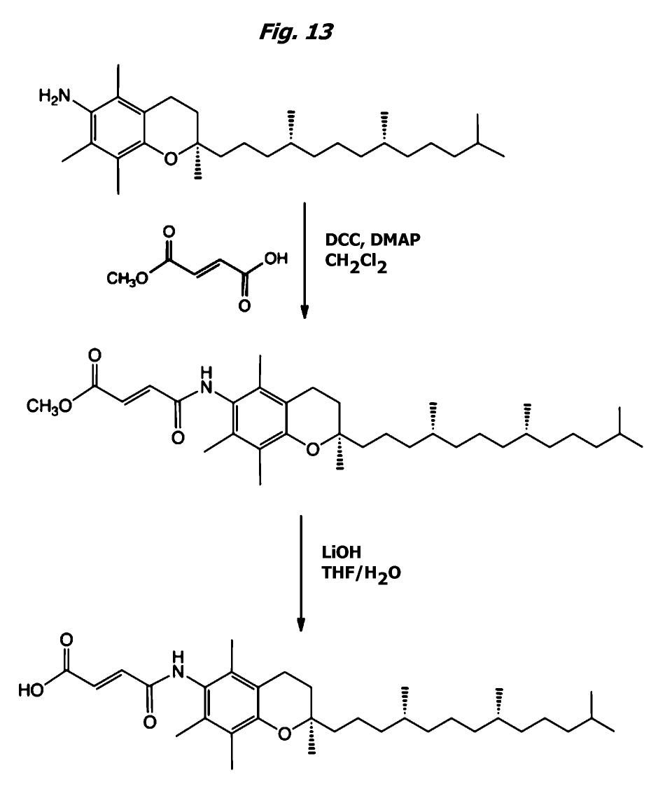

Fumaric acid as the monoester may be directly coupled to a tocopheramine using a coupling agent such as a carbodiimide in dimethylaminopyridine and dichloromethane as shown in FIG. 13. The amide is then deprotected at the terminal carboxyl using LiOH in tetrahydrofuran with water. A yellowish solid is purified chromatographically and found to be α-tocopheryl fumaramide.

This synthetic route (FIG. 13) may also be used to make the oxalamide and the malonamide by substitute the monomethyl ester of the appropriate diacid. It is to be used when the cyclic anhydride precursor of the dicarboxylic acid is not commercially available or is too strained to be stable. The synthetic route may also be adapted to make the hemi-carboxylic acid malamide, for example using a commercially available diprotected malic acid incorporating a dioxolane.

A comparison of the properties of selected members of the class is presented in FIGS. 14A and 14B. In FIG. 14A, esters and amides of the chromanol and N-chroman genera are compared. IC₅₀ against Meso-2 cells is tabulated (*), showing markedly increased cytolytic potency of αTAM and αTAM versus the corresponding tocopheryl succinates. There is a correlation between increased potency and CLogP, where amide lipophilicity is consistently greater than that of the corresponding esters. Other factors of molecular structure also play a role.

FIG. 14B demonstrates an effect of functional moiety (I) substitution for a tocopheramide series. Compared are α-tocopheryl oxalamide, with the lowest CLogP, and α-tocopheryl glutaramide, with the highest CLogP. Work is currently underway to correlate the size of the chain with pro-apoptotic potential in liposomal form. Cytolytic potential of bioisosteres with substitutions in the hydrophilic tail domain (III) may also be determined.

N-chromans are generally characterized by a lower IC₅₀ against cancer cell lines and may have a higher systemic toxicity when administered in solutions in ethanol or DMSO or as self-vesiculated vesicles. Together, chromans are configured to be pro-apoptotic by derivatization through a process of trial and error which includes modification of functional domain residues, modification of hydrophobic tail residues, modification of CLogP of the compounds, and substitution of bioisosteres. Efficacy and tolerance is also significantly enhanced by the process of formulation. Here production is modified by elimination of an intermediate freeze-thaw step prior to lyophilization to minimize the volume of the aqueous compartment and preserve the oligolamellar characteristics of the liposomal product. By control of composition and processing, particle size may be controlled in the range of 20 to 180 nm, more preferably 60 to 180 nm, and most preferably 80 to 140 nm. Zeta potential is preferably greater than −10 mV, and polydispersity indices are in the range of 0.05 to 0.15. These parameters are preferred for injectables.

Liposome mixtures of the present invention find use in therapeutic applications for the intravenous administration of the N-chromans of the genus, for example, and more generally, N-chromanyl amino-hemi-dicarboxylic acid amides and chromanols as defined herein. When reconstituted with a physiologically compatible buffer, the dry mixtures form monodisperse liposomal suspensions suitable for use as a medicinal compound.

The invention is thus also a method for manufacture of a medicament for treatment of a cancer in a mammalian host, and is characterized in that a pro-apoptotic chroman in combination with a liposome-forming lipid and a lyoprotectant excipient is formed by lyophilization into a dry solid reconstitutable as a liposomal suspension. The dry solid is thus a lyophilized liposomal aqueous suspension precursor.

N-chromans formulated in the liposomes of the present invention may include α-tocopheryl maleamide, α-tocopheryl fumaramide, α-tocopheryl succinamide, α-tocopherol malonamide (hemi-carboxylic acid of malonic acid amide), α-tocopheryl oxalamide, α-tocopheryl malamide (hemi-carboxylic acid of malic acid amide), and α-tocopheryl glutaramide (hemi-carboxylic acid of glutaric acid amide), and their tocotrienol analogs, and other bioisosteres thereof.

N-chromans are characterized by a hemi-dicarboxylic acid residue that is an unsaturated dicarboxylic acid residue having 0 to 5 carbons, a saturated dicarboxylic acid residue having 0 to 5 carbons, an oxalic acid residue, a maleic acid residue, a fumaric acid residue, a butenedioic acid residue, a glutaric acid residue, a succinic acid residue, a pentenedioic acid residue, a phthalic acid residue, a terephthalic acid residue, a malonic acid residue, a malic acid residue, a glutaric acid residue, or a bioisostere thereof. Liposomes of the present invention may also be used to formulate chromanols and other bioisoteres thereof.

In earlier work, improved apoptotic potency and efficacy was achieved through the synthesis of α-tocopheryl maleamide (αTAM), an esterase-resistant analogue of α-tocopheryl maleate. In vitro tests demonstrated significantly higher cytotoxicity of αTAM towards cancer cells (MCF-7, B16F10) compared to αTOS and other analogues prone to esterase-catalyzed hydrolysis. However, in vitro models demonstrated that αTAM was cytotoxic to non-malignant cells (e.g. lymphocytes and bone marrow progenitors), and exhibited systemic toxicity caused by inappropriate physical form of αTAM when given as solvent-solubilized and self-vesiculated dosage forms. Advantageously, we have found that lyophilized liposomal formulations resolve the non-specific and generalized toxicity of αTAM (neurotoxicity and anaphylaxis), as well as the low solubility of the compounds, and are generally useful for delivery of N-chromans and chromanols.

Remarkably, neither acute toxicity nor immunotoxicity implicated in in vitro testing was detected in vivo upon administration of liposomal αTAM formulations of the invention, without loss of efficacy, a paradoxical result. By formulating the compound in a multilamellar phosphatidylcholine liposome, the selective pro-apoptotic activity was restored. Dose loading of 40 mg/kg or more was readily achieved. The compound was found to be about ten times more potent, on an equivalent dosage basis, than α-tocopheryl succinate in mice, without any sign of toxicity as assessed by the Berlin toxicity test and gross pathological examination of the animals, but only when formulated as a liposome. Improved activity is thought to be related to a higher AUC and redistribution within serum proteins.

Moreover, liposomal formulation of αTAM and αTOS both prevented the growth of tumors in transgenic FVB/N c-neu mice bearing spontaneous breast carcinomas. However, liposomal formulations of αTAM demonstrated anti-cancer activity at levels 10-fold lower than those of αTOS, well within range of clinically acceptable dosages. Thus, the liposomal formulation of αTAM preserved its strong anti-cancer efficacy while eliminating the in vivo toxicity found of the free drug applied in DMSO.

Thus in another embodiment, the invention is a method for achieving a favorable selective cytotoxicity profile for chromans by manufacture in a liposome. The invention is a method of manufacture of a medicament for treatment of cancer in a mammalian host, characterized in that a pro-apoptotic chroman is formed in combination with a polar liposome-forming lipid and a lyoprotectant excipient as a reconstitutable dehydrated liposomal suspension precursor. The pro-apoptotic chroman may be an N-chromanyl amino hemi-dicarboxylic acid amide or a bioisostere thereof, or a chromanol or a bioisostere thereof. The polar liposome-forming lipid may be a phospholipid, a lecithin, a dialkyl or monoalkyl polyglycerol ether, a phospholipid analogue, an alkyl ether, a cholesterol salt, or a combination thereof, while not limited thereto.

In another embodiment, the invention is a method for manufacture of a medicament for treatment of a cancer in a mammalian host, characterized in that a pro-apoptotic chroman in combination with a polar liposome-forming lipid and a lyoprotectant excipient is formed by lyophilization into a reconstitutable liposomal suspension precursor for intravenous injection or infusion. A step of the method is modified to minimize the volume of the sequestered aqueous compartment prior to lyophilization, that step being omission of an intermediate freeze-thaw process step prior to lyophilization. The method may also be modified to more fully preserve the oligolamellar character of the hydrated suspension so as to minimize precipitation and slow release of the chroman.

In another aspect, the invention is a liposomal formulation comprising a pro-apoptotic chroman and a polar liposome-forming lipid for treatment or prevention of a cancer in a mammalian host, where the route of administration is by intravenous administration, by instillation, by injection, or as otherwise found compatible. The polar liposome-forming lipid is a phospholipid, a lecithin, a dialkyl or monoalkyl polyglycerol ether, a phospholipid analogue, an alkyl ether, a cholesterol salt, or a combination thereof, for example. The liposome formulation may be lyophilized; the formulation is thus a liposomal suspension precursor for reconstitution prior to use. Such lyophilized mixed liposome formulations comprise a lyoprotectant excipient and the suspension is processed to be essentially water-free. The preferred lyophilized liposomal suspension precursor may be stored under argon in the dry state and has prolonged stability on storage.

In a preferred embodiment, the above formulations are found to be useful where the vitamin E analogue is an N-chroman or bioisostere thereof, or a chromanol or bioisostere thereof, wherein a tumor-selective oncolytic effect is exerted when said formulation is administered according to a dosage regime for treatment of a tumor in said mammalian host by injection, by installation or by intravenous infusion; and further wherein said formulation achieves an efficacious blood level of said pro-apoptotic chroman without pharmacologically unacceptable systemic toxicity.

Liposome-based targeted delivery systems for these various analogues of vitamin E are of utility for further development of efficient and safe drug formulations for clinical trials.

### INCORPORATION BY REFERENCE

Descriptions of certain aspects of the invention appear in published form. Publications include Turanek, J et al, 2009, Liposomal formulation of α-tocopheryl maleamide: in vitro and in vivo toxicological profile and anticancer effect against spontaneous breast carcinomas in mice. Tox Appl Pharma 237:249-57, and Koudelka S et al, 2009, Lyophilized liposome-based formulations of α-tocopheryl succinate: preparation and physico-chemical characterization, J Pharm Sci 99:2434-43), which are incorporated in full by reference as if reproduced herein.

All of the U.S. patents, U.S. patent application publications, U.S. patent applications, foreign patents, foreign patent applications and non-patent publications referred to in this specification and/or cited in accompanying submissions, are incorporated herein by reference, in their entirety. When cited works are incorporated by reference, any meaning or definition of a word in the reference that conflicts with or narrows the meaning as used here shall be considered idiosyncratic to said reference and shall not supersede the meaning of the word as used in the disclosure herein.

Although the foregoing invention has been described in some detail by way of illustration and example for purposes of clarity of understanding, it will be clear to one skilled in the art that changes, substitutions, combinations and modifications may be practiced within the scope of the appended claims. In general, in the following claims, the terms used should not be construed to limit the claims to one or more specific embodiments disclosed in the specification and the claims, but should be construed to include all possible embodiments along with the full scope of equivalents to which such claims are entitled. Therefore, the scope of the present invention shall be determined not with reference to the above description but shall, instead, be determined by the construction of the appended claims, along with their full scope of equivalents.

We claim:

1. A lyophilized liposomal formulation of a pro-apoptotic chroman, which comprises:
a) up to 30% (as mol %) of an N-chromanyl amino hemi-dicarboxylic acid amide or a bioisostere thereof;
b) a lecithin; and
c) a lyoprotectant excipient;
wherein said formulation, when reconstituted with a physiologically compatible diluent, forms a monodisperse multilamellar liposomal suspension.

2. The liposomal formulation of claim 1, wherein said N-chromanyl amino hemi-dicarboxylic acid amide is α-tocopheryl maleamide.

3. The liposomal formulation of claim 1, wherein said N-chromanyl amino hemi-dicarboxylic acid amide is α-tocopheryl succinamide.

4. The liposomal formulation of claim 1, wherein said N-chromanyl amino hemi-dicarboxylic acid amide is α-tocopheryl fumaramide.

5. The liposomal formulation of claim 1, wherein said N-chromanyl amino hemi-dicarboxylic acid amide is α-tocopheryl malonamide.

6. The liposomal formulation of claim 1, wherein said N-chromanyl amino hemi-dicarboxylic acid amide is α-tocopheryl glutaramide.

7. The liposomal formulation of claim 1, wherein said N-chromanyl amino hemi-dicarboxylic acid amide is α-tocopheryl malamide.

8. The liposomal formulation of claim 1, wherein said N-chromanyl amino hemi-dicarboxylic acid amide is α-tocopheryl oxalamide.

9. The liposomal formulation of claim 1, wherein said hemi-dicarboxylic acid is an unsaturated dicarboxylic acid residue having 0 to 5 carbons, a saturated dicarboxylic acid residue having 0 to 5 carbons, an oxalic acid residue, a maleic acid residue, a fumaric acid residue, a butenedioic acid residue, a glutaric acid residue, a succinic acid residue, a pentenedioic acid residue, a phthalic acid residue, a terephthalic acid residue, a malonic acid residue, a malic acid residue, or a bioisostere thereof.

10. The liposomal formulation of claim 1, wherein said lyoprotectant excipient is sucrose, trehalose, arabinose, erythritol, fructose, galactose, glucose, lactose, maltitol, maltose, maltotriose, mannitol, mannobiose, mannose, ribose, sorbitol, saccharose, xylitol, xylose, dextran, or a mixture thereof, said lyoprotectant excipient for forming an amorphous glass with a glass transition temperature.

11. The liposomal formulation of claim 10, wherein said lyoprotectant excipient is formulated with a plasticizer to adjust the glass transition temperature, where said plasticizer is glycerol, dimethylsulfoxide, a lower molecular weight polyethylene glycol, propylene glycol, diethylene glycol dimethylether, triethyleneglycol dimethyl ether, tetraethylene glycol dimethyl ether, N,N-dimethylacetamide, N,N-dimethylformamide, tetramethyurea, or a mixture thereof.

12. The liposomal formulation of claim 9, wherein said lyoprotectant excipient is formulated with a binder, where said binder is polyvinylpyrrolidinone, high molecular weight polyethylene glycol, a block copolymer of polypropyleneglycol and polyethylene glycol, polyacrylate, polymethylmethacrylate, poly-(d-1-lactide-co-glycolide), triethylene glycol dimethylether, butyl diglyme, chitosan, a cellulose, a methylcellulose, an alginate, an albumin, a dextran, a starch, a gelatin, or mixtures thereof.

13. The liposomal formulation of claim 1, further comprising a stealth coat of a polyethyleneglycol or a hyaluronic acid.

14. The liposomal formulation of claim 13, wherein said polyethyleneglycol is tocopheryl polyethyleneglycol succinate, pegylated dipalmitoyl phosphatidylcholine, a pegylated phosphatidyl glycerol, a pegylated phosphatidyl-ethanolamine, or a pegylated ceramide.

15. The liposomal formulation of claim 1 having a polydispersity of less than 0.2, and more preferably less than or about 0.15, when reconstituted with a physiologically compatible diluent.

16. The liposomal formulation of claim 1, wherein said N-chromanyl amino hemi-dicarboxylic acid amide has a CLogP of less than or about 11.

17. The liposomal formulation of claim having a mean size of 20 to 180 nanometers, more preferably 50 to 140 nanometers, when reconstituted with a physiologically compatible diluent.

18. The liposomal formulation of claim 1, wherein said lyophilized liposomal formulation is stable for at least 6 months under refrigeration.

19. A method for manufacture of a medicament for treatment of cancer in a mammalian host, characterized in that a pro-apoptotic chroman in combination with a polar liposome-forming lipid and a lyoprotectant excipient is formed by a lyophilization step as a lyophilized multilamellar liposomal suspension precursor formulation.

20. The method of claim 19, wherein said polar liposome-forming lipid is a phospholipid, a lecithin, a dialkyl or monoalkyl polyglycerol ether, a phospholipid analogue, an alkyl ether, a cholesterol salt, or a combination thereof.

21. The method of claim 19, where a step of the method is modified to minimize the volume of the sequestered aqueous compartment of said multilamellar liposomal suspension prior to lyophilization, said step being omission of an intermediate freeze-thaw process step prior to lyophilization.

22. The formulation of claim 19, wherein a tumor-selective oncolytic effect is exerted when said lyophilized multilamellar liposomal suspension precursor formulation is reconstituted and administered according to a dosage regime for treatment of a tumor in said mammalian host by injection, instillation, or by intravenous infusion; and further wherein said formulation achieves an efficacious blood level of said pro-apoptotic chroman without pharmacologically unacceptable systemic toxicity.
